(19) United States
(12) Patent Application Publication
(10) Pub. No.: US 2023/0116248 A1
(43) Pub. Date: Apr. 13, 2023

(54) LIGHT APPLICATOR FOR THE EXECUTION OF A TRANSCUTANEOUS PHOTODYNAMIC THERAPY (PDT)

(71) Applicant: Richard Wolf GmbH, Knittlingen (DE)

(72) Inventors: Bernd Claus WEBER, Karlsruhe (DE); Stephan SIEBER, Knittlingen-Freudenstein (DE)

(21) Appl. No.: 17/960,187

(22) Filed: Oct. 5, 2022

(30) Foreign Application Priority Data
Oct. 7, 2021 (DE) 10 2021 211 330.3

(51) Int. Cl. A61N 5/06

(52) U.S. Cl. CPC A61N 5/0616; A61N 5/062; A61N 2005/0644

(57) ABSTRACT

A light applicator (1) executes a transcutaneous photodynamic therapy (PDT), in tissue (25) of an organic body (23) and includes a needle section (5), extending longitudinally along an insertion axis (L), and at least one light-emitting element (7) at the distal end (3) of the needle section (5). An at least partially light-transparent applicator tip (9) extends at least distally from the at least one light-emitting element (7), for insertion of the needle section (5) into the tissue (25) of the organic body (23) along the insertion axis (L). A handgrip element (19) is arranged proximally with respect to the needle section (5) for manual positioning of the light applicator (1). The handgrip element (19) can be coupled to the needle section (5) for positioning and/or insertion, and is configured to be detachable from the needle section (5) for the execution of the PDT.

### CROSS REFERENCE TO RELATED APPLICATIONS

This application claims the benefit of priority under 35 U.S.C. § 119 of German Application 10 2021 211 330.3, filed Oct. 7, 2021, the entire contents of which are incorporated herein by reference.

### TECHNICAL FIELD

The present disclosure relates to a light applicator for the execution of a transcutaneous photodynamic therapy (PDT).

### TECHNICAL BACKGROUND

PDT is a well-known medical therapy for pathological tissue of a patient. For this purpose, a photosensitizer or marker substance is administered to the patient that selectively accumulates on the pathological tissue to be treated. In the PDT, light is then applied directly onto, or even into, the pathological tissue by means of a light applicator, or a plurality of light applicators, in order to promote the light-induced formation of oxygen radicals by means of the locally enriched photosensitizer or marker substance and thereby destroy the pathological tissue, such as a tumor. Typically, laser light is coupled into a light guide for this purpose, and directed onto the tissue. If the pathological tissue is located over an area of an external surface of the body, for example, the skin, or an internal surface, for example, the inner surface of the esophagus or intestinal walls, then it is relatively easy to extract the therapy light and radiate it onto the pathological tissue surface. However, if the pathological tissue extends over a volume in an internal organ or organ segment, it is not always possible to effectively irradiate a tumor from the “outside”, on account of the limited depth of penetration of the light into the tissue. In such cases, the PDT is particularly effective if the light is irradiated from inside the pathological tissue volume. For this purpose, the light applicator(s) must be inserted into the pathological tissue. This is also known as interstitial (through internal surfaces) and/or transcutaneous (through the skin) PDT. U.S. Pat. No. 6,048,359, for example, describes a system for the execution of a transcutaneous PDT so as to irradiate an internal volume within the body of a patient. Here a number of needle-like light applicators are inserted in parallel through the skin into the body, wherein each light applicator has lateral light extraction sites distributed along its length so as to irradiate a volume in the body.

The disadvantage of this solution of known art is that the light applicators must be relatively thick, and are relatively complex and expensive, so that they cannot be implemented as disposable articles for one-time use. Moreover, the shape of the light applicators is not ergonomically adapted to manual manipulation, whereby the latter therefore requires a high degree of practice, experience and skill on the part of the operator.

### SUMMARY

This gives rise to the object of providing a more cost-effective light applicator for the execution of a transcutaneous PDT, wherein the light applicator on the one hand is easier to manipulate, and on the other hand is safer, and, if required, can be used in close proximity to other light applicators.

In accordance with the present disclosure, a light applicator for the execution of a transcutaneous photodynamic therapy, PDT, in tissue of an organic body, is provided for the solution of this problem, wherein the light applicator comprises:
a needle section extending longitudinally along an insertion axis,
at least one light-emitting element at the distal end of the needle section,
an at least partially light-transparent applicator tip extending at least partially distally from the at least one light-emitting element for purposes of insertion of the needle section into the tissue of the organic body along the insertion axis, and
a handgrip element arranged proximally with respect to the needle section, for purposes of manual positioning of the light applicator, wherein the handgrip element can be coupled to the needle section for purposes of positioning and/or insertion, and is configured to be detachable from the needle section for the execution of the PDT.

The detachable handgrip element allows ergonomic manipulation by an operator during positioning and/or insertion. In particular, during insertion, a relatively large manual force may have to be exerted by the operator onto the thinnest possible needle section. Since the handgrip element is configured to be detachable, it can be configured to be large enough to be easily grasped by the whole hand and multiple fingers. By this means a plurality of light applicators can be used together, and can be inserted relatively close to one another without the handgrip elements interfering with each other during the PDT. In certain examples of embodiment, it is even possible to use one handgrip element for purposes of positioning and/or inserting a plurality of light applicators. Moreover, the relatively bulky handgrip element, once detached, does not exert a weight force acting laterally on the needle section during the PDT if the insertion axis is not vertical, which could lead to a relatively large lateral force on the inserted applicator tip as a result of a leverage effect. Such a lateral force on the inserted applicator tip must be avoided, or minimized as far as possible, to protect the tissue of the patient, and/or the applicator tip.

The light applicator can optionally also have a connecting cable for purposes of supplying the light applicator with light and/or power by means of a light and/or power supply unit. The light-emitting element can be passively configured as a site of optical fibre light extraction from an optical fibre extending lengthwise through the needle section. The connecting cable can then have an optical fibre bundle, and the power supply unit can couple laser light into the optical fibre bundle that is connected to the latter. Preferably, however, the light-emitting element is actively configured, for example as an LED, and actively converts electrical power into light at the distal end of the needle section. Compared to an optical fibre light extraction site, an active light-emitting element, e.g. an LED, at the distal end of the needle section, has the great advantage that no expensive laser is required, the light of which must be coupled into the optical fibre by means of the power supply unit. Since the power supply unit only has to supply the light applicators with power, it can be configured in a particularly simple and inexpensive manner. The active light-emitting element preferably has a light spectrum matched to the photosensitizer or marker material. Alternatively or additionally, a light filter can be used for this purpose.

The connecting cable can optionally have a proximal-side connector for purposes of connecting to a port on a power supply unit. The power supply unit preferably has a number of ports, in order to be able to connect and supply power and/or light simultaneously to a number of light applicators.

The connecting cable can optionally have a distal-side connector, wherein the distal-side connector is configured to be detachable from the needle section for purposes of positioning and/or insertion, and can be connected to a proximal-side connector of the needle section for the execution of the PDT. This can be useful to keep the connecting cable completely separate from the needle section for purposes of positioning and/or insertion, and to connect it to the distal-side connector only when the needle section is finally positioned and inserted, when it is needed for the PDT.

The proximal-side connector of the needle section can optionally be protectively enclosed by the handgrip element when the latter is coupled on, and the distal-side connector of the connecting cable can only be connected to the proximal-side connector of the needle section when the handgrip element is detached. By this means, the proximal-side connector of the needle section is protected from damage and contamination until it is needed for purposes of connecting the connecting cable.

As an alternative to a distal-side connector of the connecting cable and a corresponding proximal-side connector of the needle section, the connecting cable can be fixedly connected to the needle section on the distal side. This has the great advantage that the weight and size of the proximal end of the needle section can be reduced, because an additional plug and socket for purposes of connecting the connecting cable to the connector of the needle section can be dispensed with at this point. This makes it possible to minimize any weight forces acting laterally with a leverage effect if the insertion axis is not vertical. The operator is also spared the need for further connections, which can significantly shorten the preparation time for the PDT, especially when a large number of light applicators are to be used. Any incorrect connection of the connecting cable to the needle section is also avoided.

The handgrip element can optionally have a first length along the insertion axis and the connecting cable can have a second length, wherein the second length is many times greater than the first length. A sufficiently long connecting cable is advantageous in order to have as much freedom as possible when positioning the light applicator with respect to the power supply unit. On the other hand, long connecting cables can lead to a tangle of cables and/or a lack of clarity when many light applicators are used in parallel. Moreover, a connecting cable that is too long can sag unhygienically onto the floor, or come into contact with other non-sterile objects. Finally, the connecting cable, due to its own weight, and/or tensile stress, can exert a lateral force on the needle section with a leverage effect, which should be minimized as much as possible. A connecting cable that is fixedly connected to the needle section is therefore preferably arranged to be as light as possible, and bundled in an orderly manner in or on the handgrip element.

For purposes of positioning and/or insertion, the connecting cable can optionally be at least partially stowed in a cavity of the handgrip element. Compared to a cable bundle arranged outside the handgrip element, a hollow cavity in the handgrip element for purposes of stowing the connecting cable has the advantage that the connecting cable is stored in a hygienically safe manner and does not disturb the operator during the positioning and/or insertion of the light applicator. The connecting cable is less exposed to bending and kinking, and can therefore be configured to be thinner and thus lighter, if it does not “dangle” outside the handgrip element as a cable bundle.

The handgrip element can optionally have at least one stowage element in the cavity, around which the connecting cable is wound in an orderly manner, and/or stowed in a serpentine arrangement. The stowage element can, for example, be a spindle running in the hollow cavity of the handgrip element, preferably along the insertion axis, around which the connecting cable is wound in a helical shape, for example. The hollow cavity is also useful for keeping the weight of the handgrip element as low as possible. The stowage element, such as a spindle, can also be configured to be hollow, if possible, in order to reduce weight.

The connecting cable can optionally be spiraled, that is to say, thermally pre-treated in such a way that it assumes a helical shape in the relaxed state, which is also reassumed each time it changes from the stretched state to the relaxed state. With this measure, the possibility can be avoided that the connecting cable rests on what may be an unhygienic surface if the distance between the patient and the power supply unit is proportionately short. The spiraled connecting cable can preferably be wound helically onto a stowage element extending axially in a hollow cavity of the handgrip element.

The proximal-side connector of the connecting cable can optionally be integrated into the handgrip element, so that the handgrip element, when removed from the needle section for the execution of the PDT, serves as a plug element for purposes of insertion into the port on the power supply unit. The proximal-side connector of the connecting cable is preferably integrated in the handgrip element on the proximal side, so that the handgrip element can be inserted in the axial direction into the port on the power supply unit for purposes of connecting the connecting cable. By detaching and removing the handgrip element from the needle section, the connecting cable stowed in the cavity of the handgrip element can be unwound as far as required. The cavity preferably has an opening on the distal side, which, when the needle section is coupled in, is closed by the proximal end of the needle section. After detaching the handgrip element from the needle section, the connecting cable can be pulled out of the handgrip element through the now open distal-side opening of the cavity.

The light applicator can optionally also comprise a guide element, which is fixedly connected to, or integrated in, the needle section at a proximal end of the needle section, and is shaped to correspond to the handgrip element in such a way that the guide element and the handgrip element can be inserted into each other in a spring-loaded manner. Such a coupling between the needle section and the handgrip element can, on the one hand, be released, and on the other hand, is strong enough, even when relatively high manual force is applied to the handgrip element, to be able to position and/or insert the needle section with as little play as possible. The coupling can preferably be released along the insertion axis by pulling the handgrip element in the proximal direction while the guide element is held in place. Locking elements can also be provided for this purpose, which can be brought into an open position, for example, by pushing laterally on the handgrip element in order to release the coupling.

The needle section can optionally have a first diameter transverse to the insertion axis, and the hand grip element can have a second diameter transverse to the insertion axis, wherein the second diameter is many times larger than the first diameter. The ergonomics for positioning and/or insertion are greatly improved if the handgrip element fits well in the whole hand for an optimal transfer of manual force. For minimally invasive PDT, on the other hand, the needle section must be as thin and rigid as possible. The first diameter of the needle section can, for example, be 1 mm or less. The second diameter of the handgrip element, on the other hand, can be 3 cm or more.

The guide element can optionally have a third diameter transverse to the insertion axis, wherein the third diameter is larger than the first diameter and smaller than the second diameter. This is useful so as to provide a secure form fit for the releasable coupling between the guide element and the handgrip element, in which the handgrip element at least partially grips around the guide element.

The handgrip element can optionally be made of at least two interconnected parts. Although the parts can be non-releasably connected to each other, this is particularly advantageous in manufacture, in order to store the connecting cable in an orderly manner in a cavity of the handgrip element. A first part of the handgrip element can form an outer wall around a cavity in the form of a sleeve part, which is open on the distal side and initially also on the proximal side. The second part can be a proximal-side cover part with a spindle extending into the cavity, on which spindle the connecting cable is wound. The cover part can then be welded or adhesively bonded to the sleeve part in such a way that the proximal-side opening of the cavity of the sleeve part is closed. The cover part preferably forms the proximal-side connector of the connecting cable for purposes of insertion into the power supply unit.

The light applicator can optionally also have electronics for light applicator identification, wherein the electronics for light applicator identification are arranged in the handgrip element. This is particularly useful in order to ensure an automatically correct power supply to the light applicator by means of the power supply unit. The selection of possible operating parameters can be reduced, or completely eliminated, if the power supply unit identifies the light applicator and reduces the number of operating parameters that can be selected correspondingly, or totally specifies them without any options. This reduces the risk of an incorrect setting of operating parameters. In particular, if the light applicator is preferably configured as a disposable item for one-time use, the light applicator identification can indicate whether the light applicator has already been used, and grant or refuse operation accordingly. The use of unauthorized light applicators can also be refused in this manner. The handgrip element with integrated connector for the connecting cable offers sufficient volume to accommodate the electronics for light applicator identification.

The handgrip element can optionally have a part that is tapered on the distal side. This is particularly useful if a number of light applicators have to be positioned particularly closely in parallel to each other, and the ergonomically-shaped handgrip element does not fit between the light applicators. For this purpose, the handgrip element can taper in the distal direction in the form of a grommet so that the part of the handgrip element that is tapered on the distal side can preferably encompass a proximal end of the needle section, when the handgrip element is coupled to the needle section for purposes of positioning and/or insertion.

In what follows, the system herein disclosed is explained in more detail with reference to the accompanying figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic representation of a light applicator for the execution of percutaneous PDT;

FIG. 2 is a schematic representation of a light applicator inserted in an organic body for the execution of percutaneous PDT;

FIGS. 3a and 3b are schematic representations of the problems of a light applicator with an ergonomically-shaped handgrip;

FIGS. 4a and 4b are schematic representations of an example of a form of embodiment of a light applicator herein disclosed;

FIGS. 5a and 5b are schematic representations of an example of a further form of embodiment of a light applicator herein disclosed;

FIGS. 6a and 6b are schematic representations of an example of a further form of embodiment of a light applicator herein disclosed;

FIGS. 7a and 7b are schematic representations of an example of a further form of embodiment of a light applicator herein disclosed;

FIGS. 8a and 8b are schematic representations of an example of a further form of embodiment of a light applicator herein disclosed;

FIGS. 8c, 8d and 8e are schematic representations of an example of a spiraled connecting cable in different states of extension;

FIGS. 9a and 9b are schematic representations of an example of a further form of embodiment of a light applicator herein disclosed; and

FIGS. 10a, 10b and 10c are schematic representations of an example of a further form of embodiment of a light applicator herein disclosed.

### DESCRIPTION OF PREFERRED EMBODIMENTS

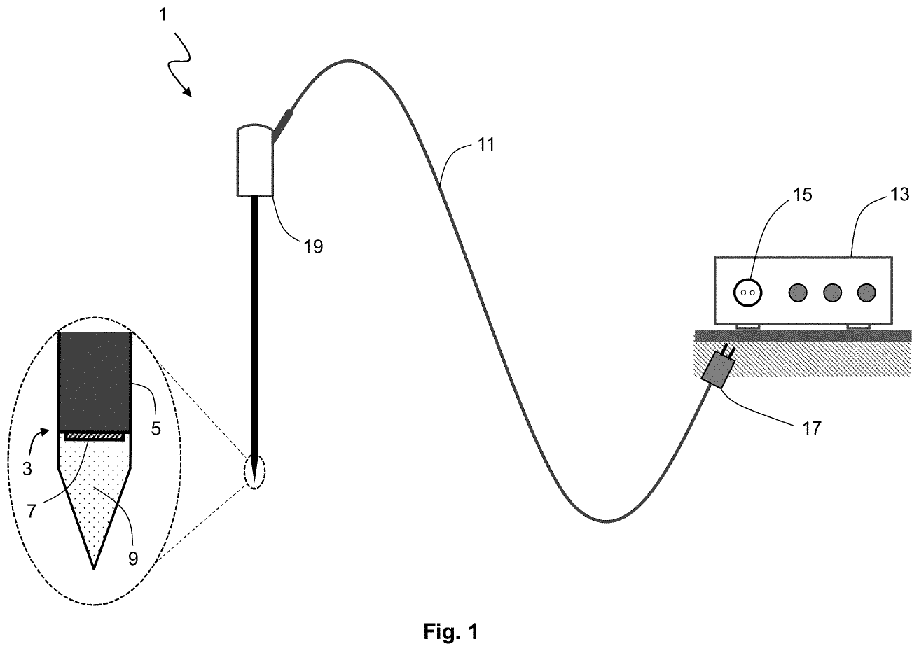

Referring to the drawings, FIG. 1 shows a needle-like light applicator 1 for the execution of a transcutaneous photodynamic therapy (PDT), which can be inserted through the skin of a patient, that is to say, transcutaneously, into pathological tissue of the patient. The light applicator 1 is as rigid and thin as possible in order to cause as little damage as possible with the insertion, that is to say, to be minimally invasive to the patient's healthy tissue. An active light-emitting element 7 in the form of an LED is arranged at the distal end 3 of a thin shaft-form needle section 5 of the light applicator 1. On the distal side of the LED 7, a light-transparent and light-diffusing applicator tip 9 is arranged at the distal end 3 of the needle section 5 of the light applicator 1. The applicator tip 9 emits the light of the LED 7 as isotropically as possible within a solid angle of more than 3π. The light radiation from the applicator tip 9 is therefore approximately spherical.

The light applicator 1 can be connected to a power supply unit 13 via a connecting cable 11, with which the light applicator 1 is supplied with power that operates the LED 7. The power supply unit 13 has a port 15 for a proximal-side connector 17 of the connecting cable 11. The light applicator 1 has a handgrip element 19 at the proximal end of the needle section 5, with which an operator can manually grip, position, and insert the light applicator 1 into the patient. The light applicator 1 can be connected and supplied with power via the connecting cable 11 with the proximal-side connector 17, which fits into the port 15 on the power supply unit 13. The needle section 5 of the light applicator 1 preferably has an electrically conductive core and an electrically conductive sheath, electrically insulated from the core, such that the core and sheath can act as a forward and return pair of conductors in order to supply power to the LED 7. Alternatively or additionally, an extra conductor can be provided in the needle section 5 of the light applicator 1.

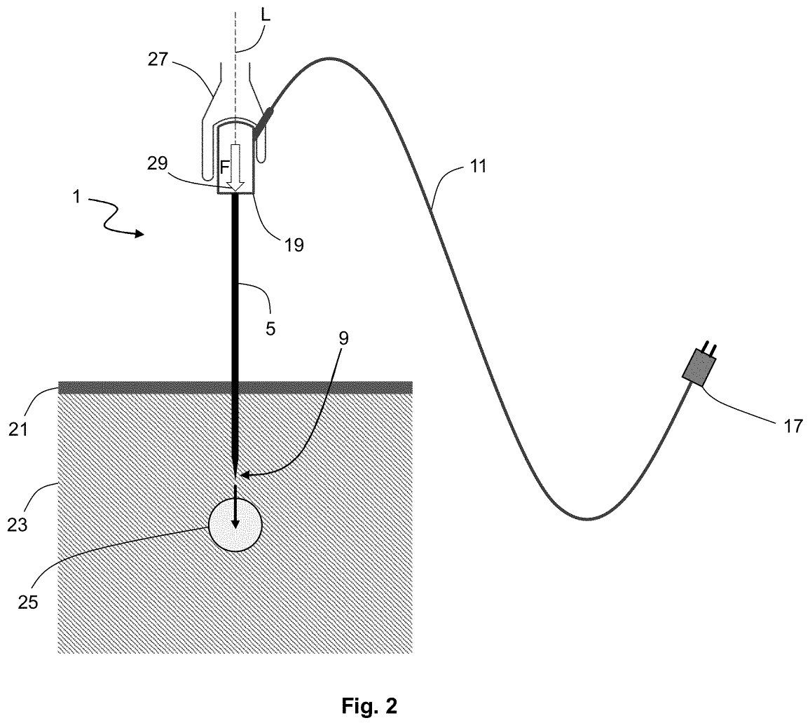

FIG. 2 shows the light applicator 1 when inserted through the skin 21 of a patient's body 23, with the objective of treating pathological tissue 25 in the patient with percutaneous PDT. For this purpose, the operator grips the handgrip element 19 with their hand 27, and exerts an insertion force 29 onto the needle section 5, directed along an insertion axis L in the distal direction.

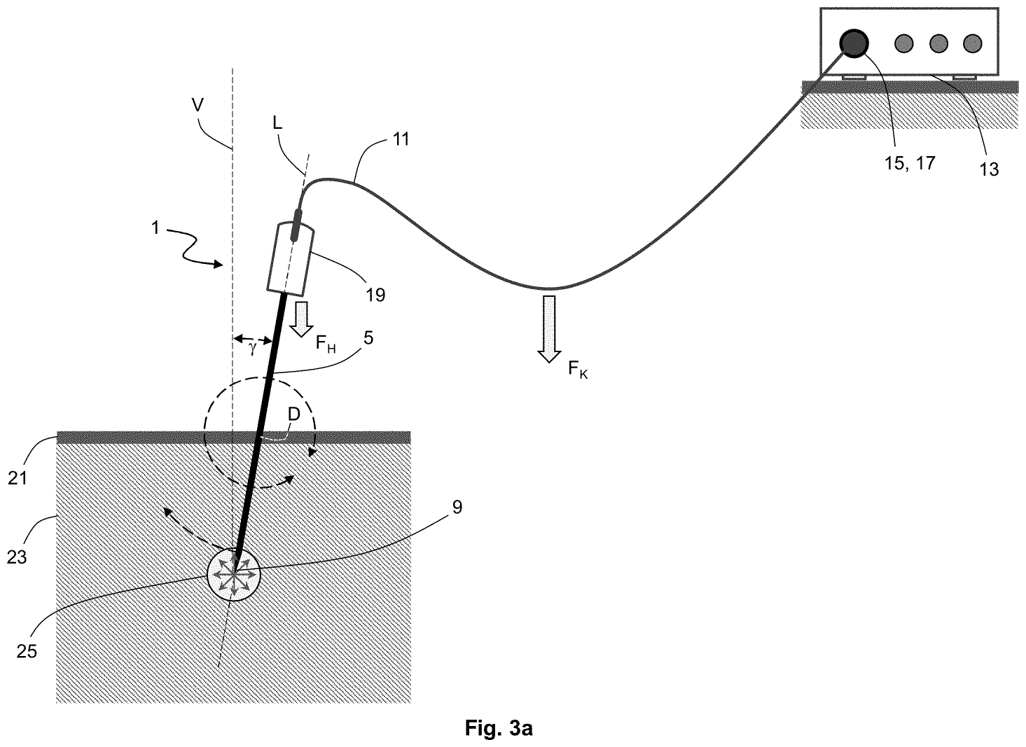

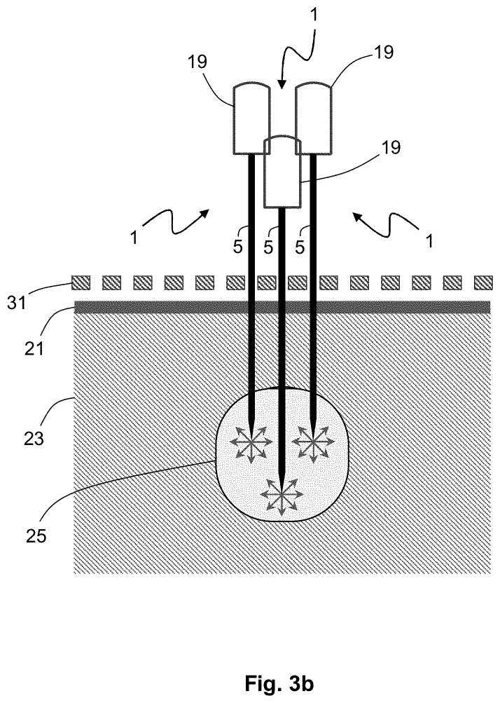

FIG. 3a shows a first problem of lateral forces, which act on the light applicator 1 by way of a leverage effect, once the light applicator 1 is finally positioned, and inserted and released, in order to execute the PDT. The applicator tip 9 is inserted into the pathological tissue 25 so as to irradiate the latter with light from within. In this example, the insertion axis L runs at an angle to the vertical V, so that the weight force FH of the handgrip element 19 exerts a torque on the inserted needle section 5, about an axis of rotation D running parallel to the skin. This torque about the axis of rotation D leads to an undesirable lateral force on the inserted part of the needle section 5, which exerts a load on the tissue. This is not only detrimental to the tissue, but in the worst case can cause the applicator tip 9 to break off. The leverage effect is all the greater, the shorter the inserted part of the needle section 5 is compared to that part of the needle section 5 located outside the body 23. In addition to the weight force FH of the handgrip element 19, a weight force FK acts on the connecting cable 11 connected to the power supply unit 13, if it does not rest in an unhygienic manner on a surface, and, as shown, is stretched through the air up to the power supply unit 13. This force also amplifies the undesirable torque on the light applicator 1 about the axis of rotation D with a relatively large leverage effect.

FIG. 3b shows a second problem when using a plurality of light applicators 1, preferably with a positioning template 31, in which the light applicators 1 are guided in a defined manner. If a close-coupled PDT is to be carried out with a plurality of light applicators 1 in what is in this case the more voluminous pathological tissue 25, the laterally protruding handgrip elements 19 of the respective light applicators 1 can interfere with each other.

In order to provide an ergonomically easy-to-manipulate handgrip element on the one hand and on the other hand to avoid or limit the problems shown in FIGS. 3a,b, the handgrip element 19 is configured to be detachable from the needle section 5 in the examples of embodiment described below.

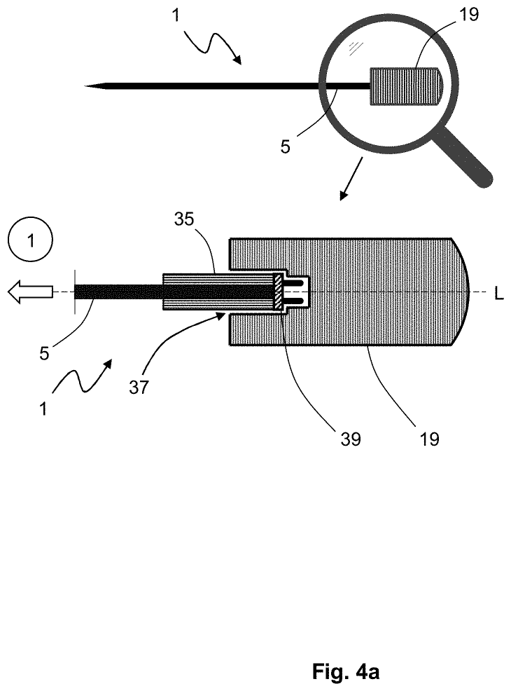

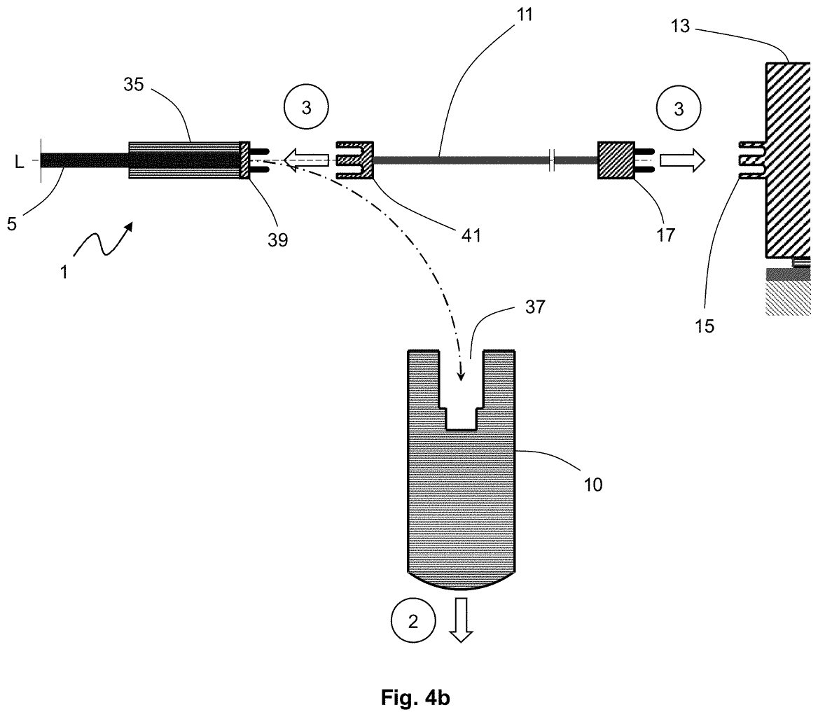

FIG. 4a shows schematically a longitudinal section of the light applicator 1 in which the handgrip element 19 is coupled onto the proximal end of the needle section 5. In this coupled configuration, the light applicator can be manually positioned and inserted as if the handgrip element 19 were fixedly connected to the needle section 5. At the proximal end of the needle section 5, the latter is of somewhat thicker configuration, or is fitted with a guide element 35 in the form of a guide sleeve.

The guide element 35 is inserted in a form-fit and/or a force-fit into a distal-side opening 37 of the handgrip element 19. In this example of embodiment, the proximal end of the needle section 5 has a proximal-side connector 39 for purposes of connecting to a distal connector 41 (see FIG. 4b) of the connecting cable 11. In the coupled configuration shown in FIG. 4a, the handgrip element 19 protectively surrounds the proximal-side connector 39 of the needle section 5, so that the connecting cable 11 can only be connected when the handgrip element 19 is detached from the needle section 5.

Once the light applicator 1 has been positioned and/or inserted in a first step (indicated by a circled number in the figures), the handgrip element 19 can be detached in a second step, as shown in FIG. 4b. A separate connecting cable 11 can then be used to connect the light applicator 1 to a power supply unit 13, which supplies the light applicator 1 with power via the connecting cable 11. For this purpose, the distal-side connector 41 of the connecting cable 11 is connected to the proximal-side connector 39 of the needle section 5 and the proximal-side connector 17 of the connecting cable 11 is connected to the port 15 on the power supply unit 13.

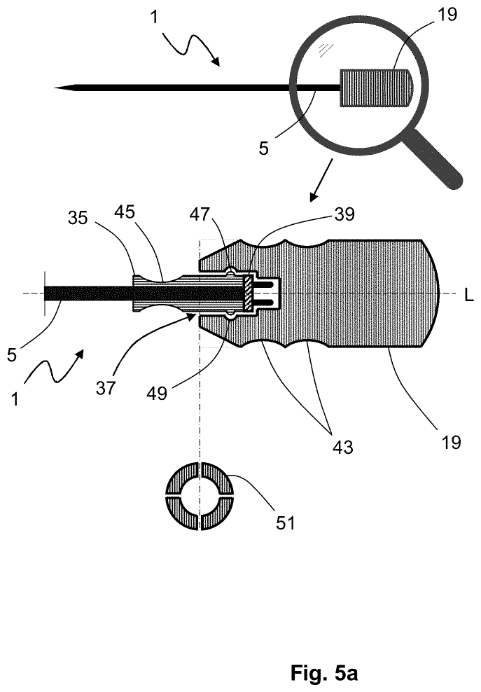

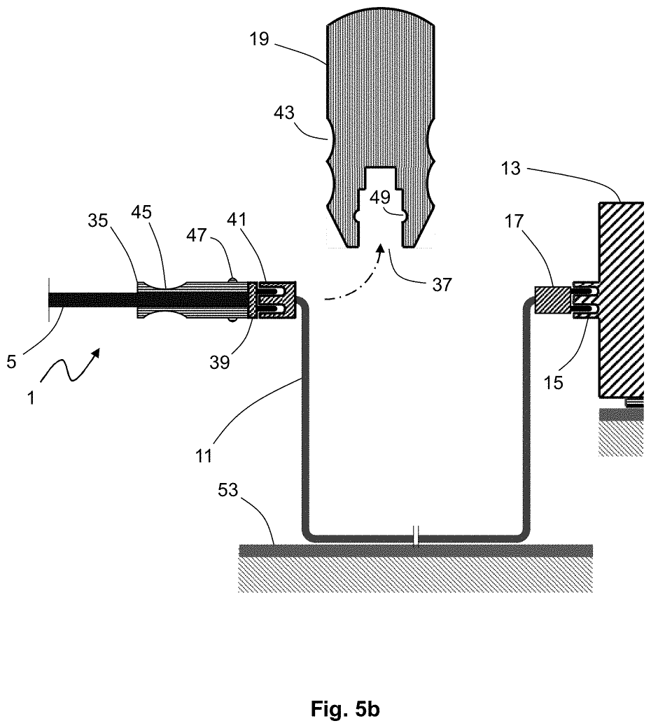

FIG. 5a shows the principle shown in FIG. 4a in a more detailed form of embodiment. Here, the handgrip element 19 has laterally arranged grip recesses 43 so that the handgrip element 19 can be gripped more easily and pushed onto, or pulled off, the proximal end of the needle section 5. Correspondingly, the guide element 35 also has a grip recess 45, so that it can be held easily when coupling and detaching the handgrip element 19. The form-fit and force-fit mechanisms for purposes of coupling are achieved here by an external bead 47 on the guide element 35, which engages in a correspondingly shaped internal receiving groove 49 in the handgrip element 19. The handgrip element 19 is slotted on the distal side, as shown in the cross-section in FIG. 5a below, whereby the handgrip element 19, which is preferably made of plastic, forms spring tongues 51, which yield radially outwards. The spring tongues 51 can be pushed outwards by the bead 47 against a spring force so as to momentarily expand the inner diameter of the distal-side opening 37 of the handgrip element 19, in order to bring the bead 47 into, and out, of the receiving groove 49. FIG. 5b shows the connecting cable 11 in the connected state, after the handgrip element 19 has been detached, which cable partially rests on a surface 53, if the distance between the patient and the light applicator 1 inserted into the latter on the one hand, and the power supply unit 13 on the other hand, is proportionately small.

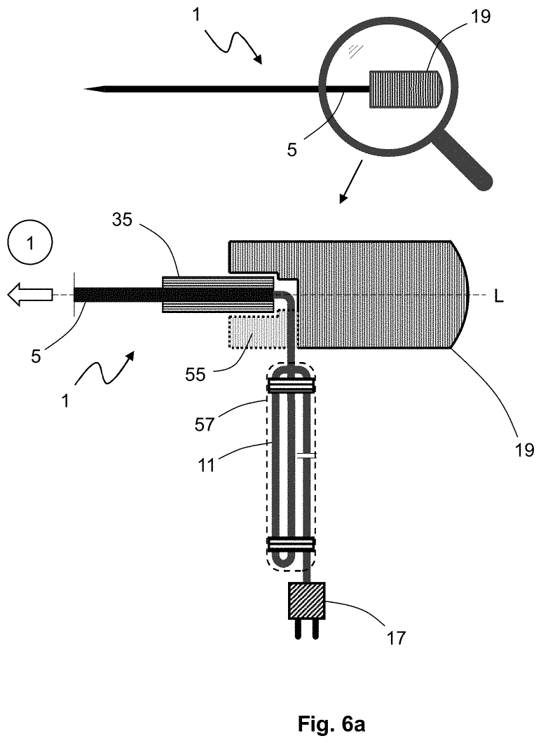

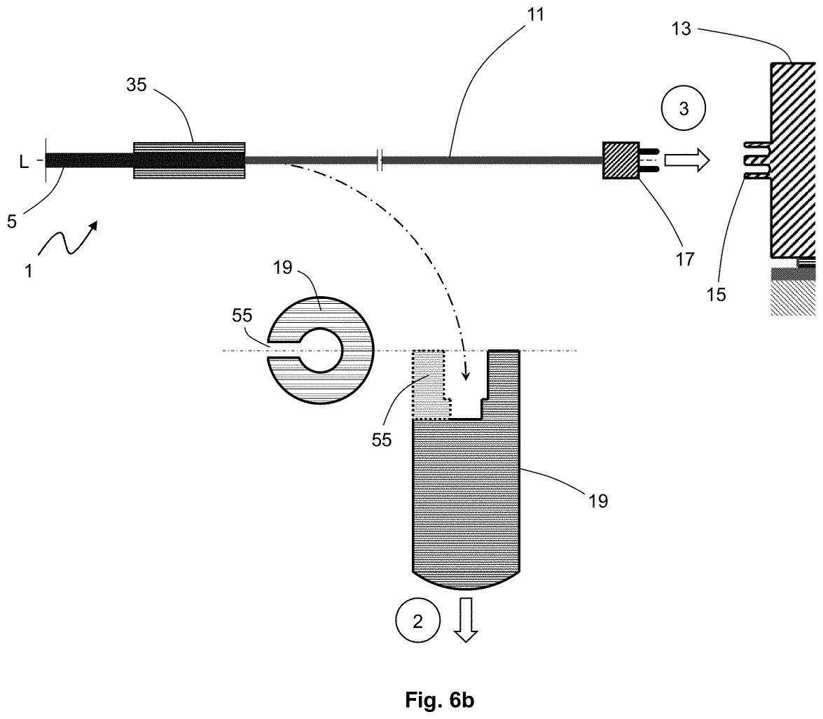

FIGS. 6a,b show an example of embodiment in which the connecting cable 11 is fixedly connected to the needle section 5. Here, therefore, there is no distal-side connector on the connecting cable 11, and no corresponding proximal-side connector on the needle section 5. The connecting cable 11 here is bent outwards through approx. 90° directly behind the needle section 5 and guided out of the handgrip element 19. For this purpose, the handgrip element 19 has a cable recess 55 that is open on the distal side.

Here the connecting cable 11 is neatly bundled as a cable bundle 57, so that it disturbs the operator as little as possible during the positioning and/or insertion of the light applicator 1. As shown in FIG. 6b, the cable bundle 57 is unbundled after the detachment of the handgrip element 19 and the connecting cable 11 is connected with its proximal-side connector 17 to the port 15 on the power supply unit 13.

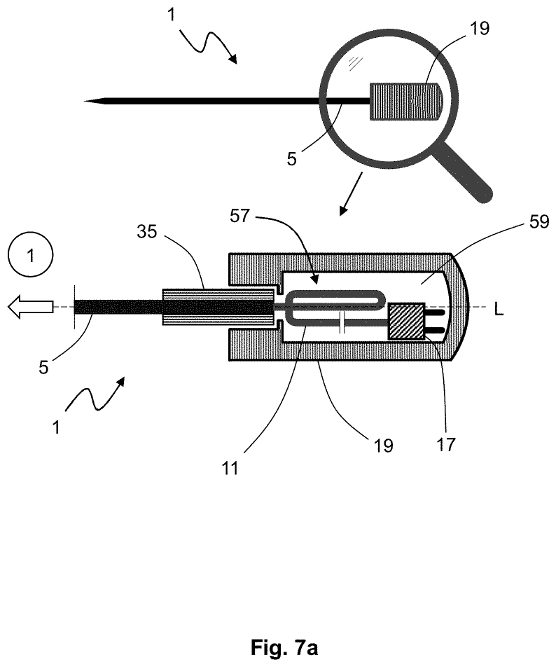

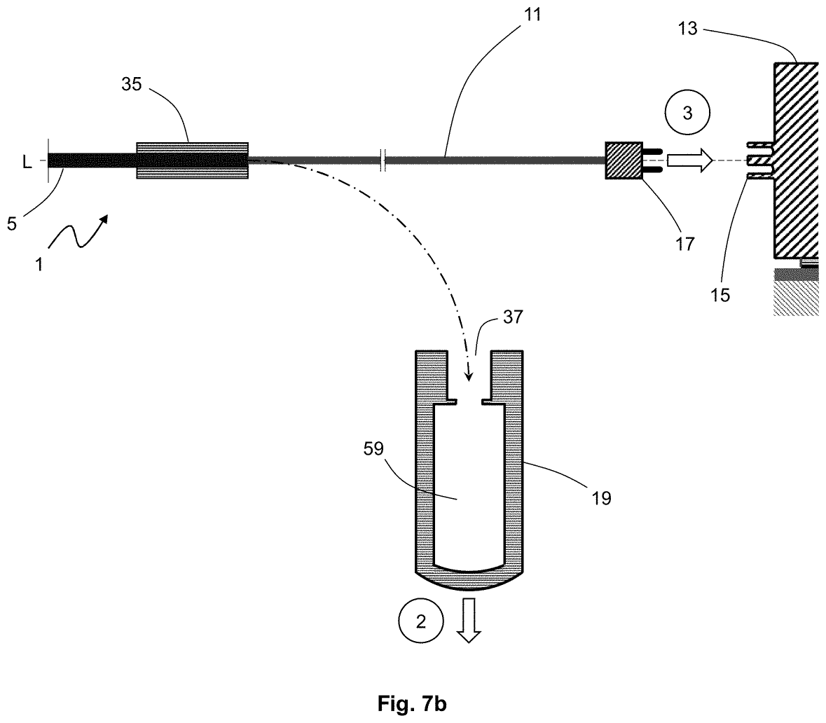

FIG. 7a shows a form of embodiment in which the cable bundle 57, together with the proximal-side connector 17 of the connecting cable 11, are stowed within a cavity 59 of the handgrip element 19. By this means, the cable bundle 57 does not disturb the operator at all during the positioning and/or insertion of the light applicator 1. Moreover, the connecting cable 11 is thus much better protected and less exposed to mechanical stresses, so that it can be configured to be thinner and lighter. As shown in FIG. 7b, after detachment of the handgrip element 19 from the needle section 5, the entire connecting cable 11, together with the proximal-side connector 17, can be pulled out of the cavity 59 through the distal-side opening 37 of the handgrip element 19, whereby it is unbundled so that it can be connected to the power supply unit 13.

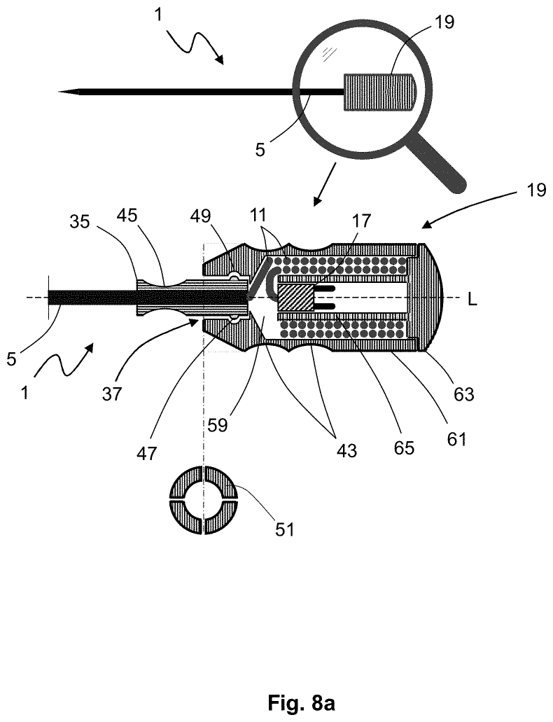

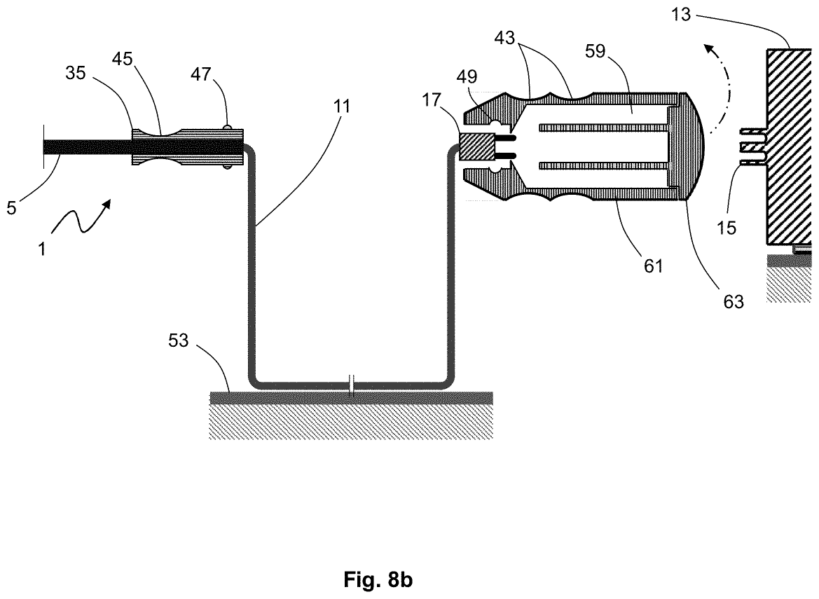

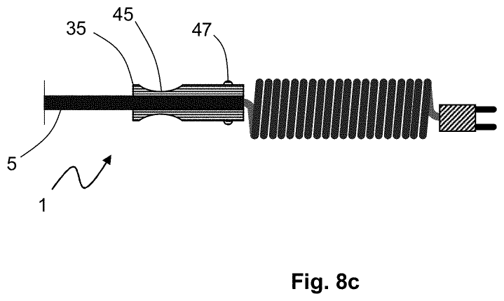

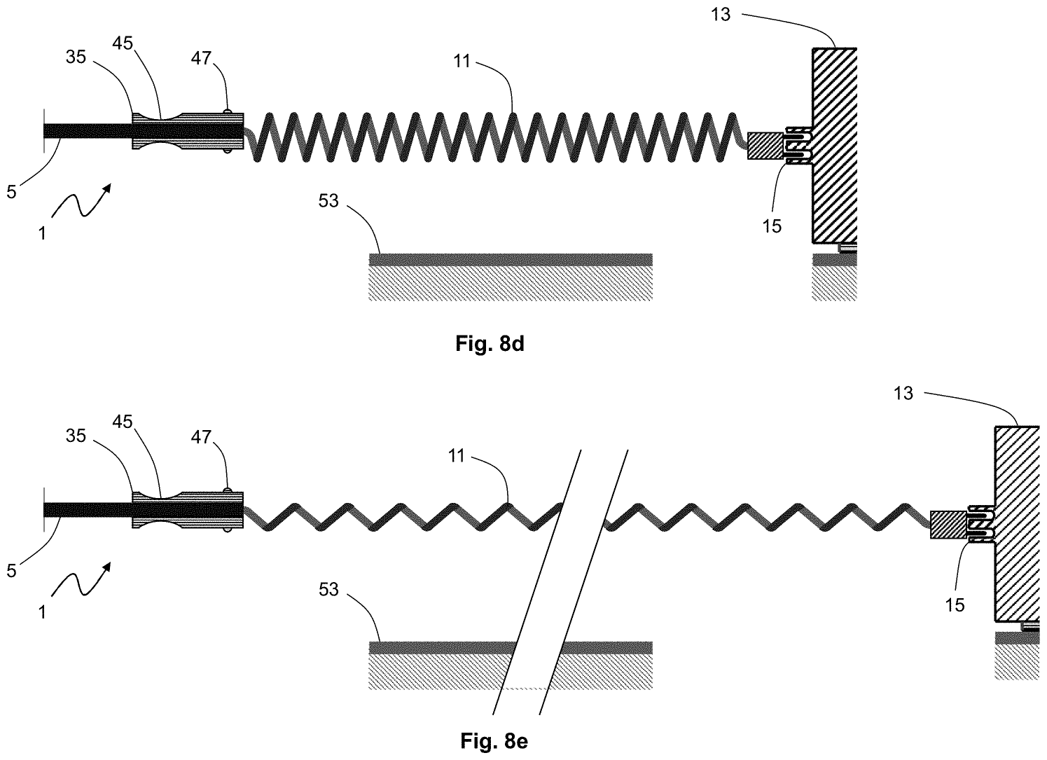

FIGS. 8a,b show the principle shown in FIGS. 7a,b in a more detailed form of embodiment analogous to FIGS. 5a,b. The coupling principle and the external shape of the handgrip element 19, as well as the guide element 35, are as in the example of embodiment shown in FIGS. 5a,b. Here, however, the handgrip element 19 is formed from two parts 61, 63 that are fixedly connected to each other. Here the first part 61 of the handgrip element 19 is a sleeve part 61, which forms an outer wall around the cavity 59, which is open on the distal side to the opening 37, and is also open on the proximal side before connection to the second part 63. The second part 63 is here a proximal-side cover part 63, with a spindle 65 extending into the cavity 59 along the insertion axis L, which spindle 65 functions as a stowage element, on which the connecting cable 11 is wound in the coupled-on configuration of the handgrip element 19. The cover part 63 is then welded or adhesively bonded to the sleeve part 61 in such a way that the proximal-side opening of the cavity 59 of the sleeve part 61 is closed. To reduce weight, the spindle 65 is configured to be hollow, which allows it to accommodate the proximal-side connector 17 of the connecting cable 11 in a space-saving manner.

In order to prevent part of the connecting cable 11 from resting on what may be an unhygienic surface 53 when the distance between the patient, that is to say, the patient's body 23 and the light applicator 1 inserted in the latter on the one hand, and the power supply unit 13 on the other hand, is proportionately small, as is the case in FIG. 8b, a connecting cable 11 can be used, which is characterized in that it is spiraled in the relaxed state. That is to say, if no force is applied to the connecting cable 11, the connecting cable 11 assumes a helical shape, wherein the cable windings preferably lie directly against each other, that is to say, touch each other, and accordingly the axial extension of the spiral formed by the connecting cable 11 is as short as possible (see FIG. 8c). In the state when the cable windings are in contact, as shown in FIG. 8c, the diameters and the lengths of the spiral formed by the connecting cable 11 on the one hand and the spindle 65 on the other hand are preferably matched to each other, such that the spiraled connecting cable 11 can be mounted on the spindle 65 in a simple manner, and the spiral also does not protrude axially.

Only by exerting and continuously increasing a force on the connecting cable 11 does the axial extension of the spiral increase, and only by this means is the actual length of the connecting cable 11 utilized more and more, which becomes necessary when the distance between the body 23 and the power supply unit 13 is proportionately large. FIGS. 8c and d show, for different distances between the inserted light applicator 1 and the power supply unit 13, how the spiral formed by the connecting cable 11 is stretched in the axial direction, and thus the actual length of the connecting cable 11 is utilized in a different manner, depending on the situation, so that the connecting cable 11 never touches the surface 53.

Such a spiraling of the connecting cable 11 can be achieved, for example, by thermally treating the latter in an appropriate manner in the coiled state, as a result of which the helical shape is fixed such that the connecting cable 11 maintains this shape in the relaxed state, and also resumes this shape in the course of transition from the stretched state to the relaxed state, that is to say, when no external force is any longer applied to the cable.

The spiraling of the connecting cable 11 should be carried out in such a way that the force required to stretch the spiral in the axial direction is greater than the weight force of the connecting cable 11, because otherwise the spiral in the present application would already be stretched by the self-weight of the connecting cable 11, and would be totally unable to maintain its advantageous short axial length.

With increasing length, and thus increasing weight, of the connecting cable 11, the spiraling is preferably implemented in such a way that the force that has to be exerted on the cable spiral to extend the cable spiral also becomes increasingly greater. However, because this force is also transferred onto the light applicator 1 as a tensile force, this should not result in the position of the latter in the body 23 no longer being maintained, and possibly being unintentionally retracted, and even pulled out of the body 23 completely.

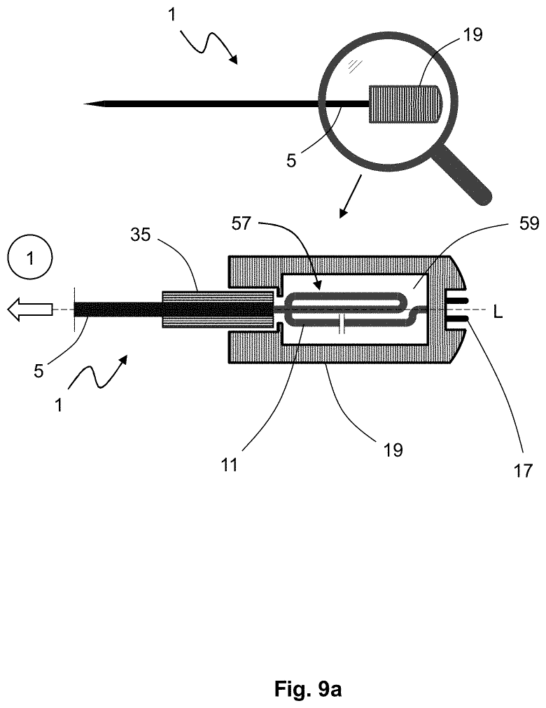

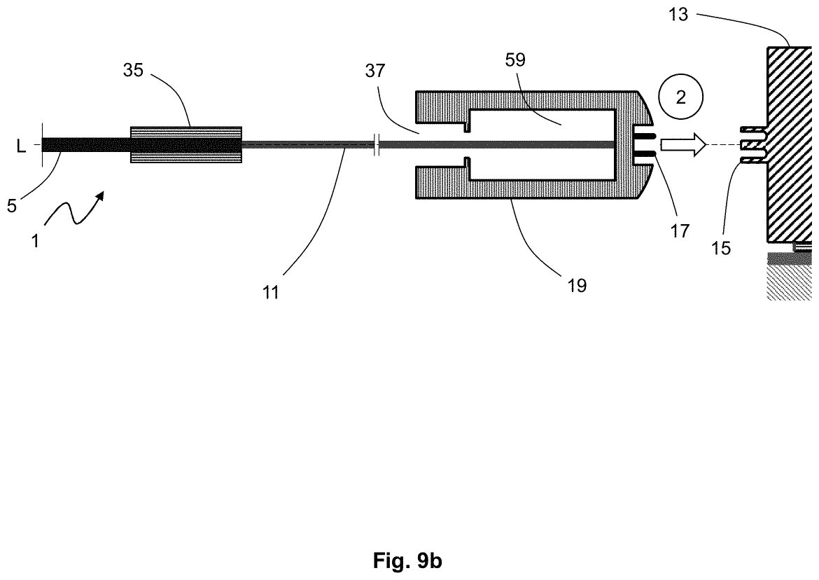

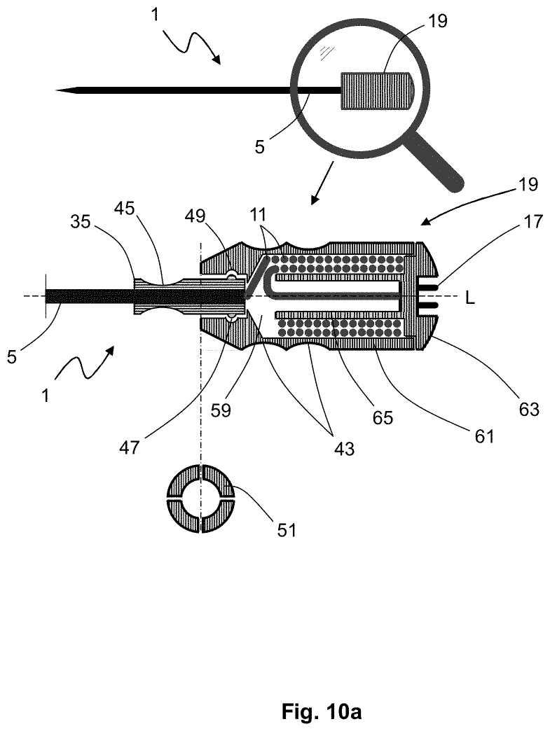

In the form of embodiment shown in FIGS. 9a,b, the proximal-side connector 17 of the connecting cable 11 is integrated into the handgrip element 19. Here, the handgrip element 19 forms, on the one hand, the proximal-side connector 17 for purposes of insertion into the port 15 on the power supply unit 13 and, on the other hand, the cavity 59 in which the connecting cable 11 is neatly stowed away as long as the handgrip element 19 is plugged onto the proximal end of the needle section 5. As shown in FIG. 9b, the handgrip element 19, when detached from the needle section 5, functions as a plug 17 for connection to the port 15 on the power supply unit 13. With the removal of the handgrip element 19 from the needle section 5, the cable bundle 57 unbundles in the cavity 59 and is pulled out of the distal-side opening 37 of the handgrip element 19 as far as is required. If the entire length of the connecting cable 11 is not required, any unutilized remainder of the cable bundle 57 can remain bundled in the cavity 59 so that the connecting cable 11 does not rest on what may be an unhygienic surface 53 (as in FIGS. 5b and 8b). Moreover, the cable arrangement is higher, which is particularly advantageous when using a plurality of light applicators 1 at the same time.

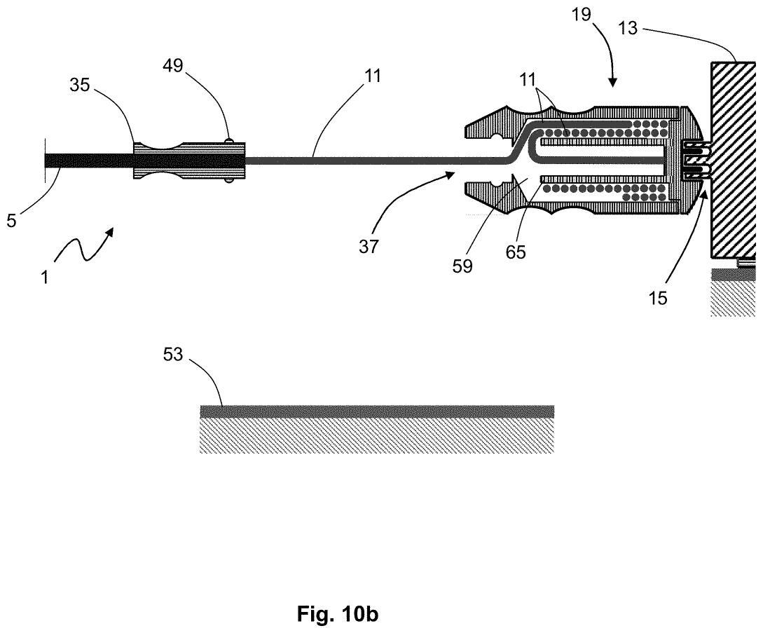

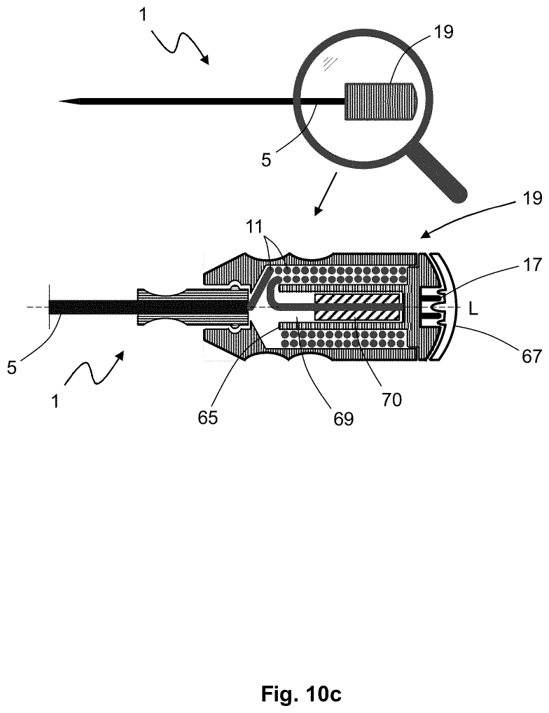

In an analogous manner to FIGS. 5a,b and 8a,b, FIGS. 10a-c show the principle shown in FIG. 9a,b in a more detailed form of embodiment. The coupling principle and the outer shape of the handgrip element 19, as well as the guide element 35, are the same as those in the examples of embodiment shown in FIGS. 5a,b and FIGS. 8a,b. In addition, the stowage of the connecting cable 11 in wound form on the spindle 65 in the cavity 59 of the handgrip element 19 is the same as that in the example of embodiment shown in FIGS. 8a,b. The example of embodiment shown in FIGS. 10a-c differs from the example of embodiment shown in FIGS. 8a,b only in that the proximal-side connector 17 of the connecting cable 11 is integrated into the handgrip element 19, namely here in the cover part 63. As shown in FIG. 10b, the handgrip element 19, when removed from the needle section 5, functions as a plug 17 for connection to the port 15 on the power supply unit 13. When the handgrip element 19 is removed from the needle section 5, the cable 11 wound onto the spindle 65 unwinds in the cavity 59 and is pulled out of the distal-side opening 37 of the handgrip element 19 only as far as required. If the entire length of the connecting cable 11 is not required, an unutilized remainder of the cable 11 wound on the spindle 65 can remain stowed in the cavity 59 so that the connecting cable 11 does not rest on what may be an unhygienic surface 53 (as in FIGS. 5b and 8b). In addition, the connecting cable 11 can be spiraled, as has been described above. This has the advantage that, for example, even if the distance between the power supply unit 13 and the body 23 into which the light applicator 1 is inserted is subsequently reduced, that is to say, if, for example, the power supply unit 13, in its already fully equipped state, is moved towards the body 23, the connecting cable 11 does not rest on what may be an unhygienic surface 53, but instead contracts into an axially-shortened spiral.

In FIG. 10c, the handgrip element 19 has a protective cap 67 that covers the integrated proximal-side connector 17 of the connecting cable 11 in a protective manner for as long as the handgrip element 19 must be held for purposes of positioning and/or insertion. The protective cap 67 can then be removed for purposes of connecting the proximal-side connector 17 of the connecting cable 11 to the port 15 on the power supply unit 13. The protective cap 67 protects both the proximal-side connector 17 of the connecting cable 11 from contamination and damage, and also the operator's hand 27 from sharp-edged pins of the connector 17. The cavity 69 formed by the hollow spindle 65 can optionally accommodate electronic components 70, for example electronic components 70 for light guide identification.

While specific embodiments of the invention have been shown and described in detail to illustrate the application of the principles of the invention, it will be understood that the invention may be embodied otherwise without departing from such principles.

### LIST OF REFERENCE SYMBOLS

1 Light applicator
3 Distal end of the needle section
5 Needle section
7 Light-emitting element/LED
9 Applicator tip
11 Connecting cable
13 Power supply unit
15 Port on the power supply unit
17 Proximal-side connector of the connecting cable
19 Handgrip element
21 Skin
23 Body
25 Pathological tissue
27 Hand of the operator
29 Insertion force
31 Positioning template
33 Positioning template guides
35 Guide element
37 Distal-side opening of the handgrip element
39 Proximal-side connector of the needle section
41 Distal-side connector of the connecting cable
43 Grip recesses
45 Grip recess
47 Bead
49 Receiving groove
51 Spring tongues
53 Surface
55 Cable recess
57 Cable bundle
59 Cavity
61 Sleeve part of the handgrip element
63 Cover part of the handgrip
65 Stowage element/spindle
67 Protective cap
69 Cavity
70 Electronic components
L Insertion axis
V Vertical
y Angle

What is claimed is:

1. A light applicator for the execution of a transcutaneous photodynamic therapy (PDT) in tissue of an organic body, the light applicator comprising:
a needle section, extending longitudinally along an insertion axis;
at least one light-emitting element at a distal end of the needle section;
an at least partially light-transparent applicator tip, extending at least partially distally from the at least one light-emitting element and configured for inserting the needle section into the tissue of the organic body along the insertion axis; and
a handgrip element, arranged proximally with respect to the needle section, and configured for manually positioning the light applicator, wherein the handgrip element is configured to be coupled to the needle section for positioning and/or insertion thereof, and is configured to be detachable from the needle section for the execution of the PDT.

2. A light applicator according to claim 1, further comprising a connecting cable configured to supply the light applicator with light and/or power by means of a power supply unit.

3. A light applicator according to claim 2, wherein the connecting cable comprises a proximal-side connector configured for connection to a port on the power supply unit.

4. A light applicator according to claim 2, wherein:
the connecting cable comprises a distal-side connector; and
the distal-side connector is configured to be detachable from the needle section for purposes of positioning and/or insertion thereof, and configured to be connectable to a proximal-side connector of the needle section for the execution of the PDT.

5. A light applicator according to claim 4, wherein:
the proximal-side connector of the needle section is protectively enclosed by the handgrip element in a state with the handgrip element coupled on the needle section; and
the distal-side connector of the connecting cable is configured to only be connected to the proximal-side connector of the needle section in a state with the handgrip element detached from the needle section.

6. A light applicator according to claim 2, wherein the connecting cable is fixedly connected to the needle section on a needle section distal side.

7. A light applicator according to claim 2, wherein:
the handgrip element has a first length along the insertion axis;
the connecting cable has a second length; and
the second length is many times greater than the first length.

8. A light applicator according to claim 2, wherein the connecting cable is at least partially stowed in a cavity of the handgrip element for positioning and/or insertion of the needle section.

9. A light applicator according to claim 8, wherein:
the handgrip element includes at least one stowage element in the cavity; and
at least one stowage element is configured for receiving the wound connecting cable, and/or stowing the connecting cable in a serpentine arrangement.

10. A light applicator according to claim 2, wherein the connecting cable is configured to have a spiraled form in a relaxed state.

11. A light applicator according to claim 10, wherein:
a stowage element extends axially in a cavity of the handgrip element; and
the connecting cable is helically wound onto the stowage element.

12. A light applicator according to claim 3, wherein:
the proximal-side connector of the connecting cable is integrated into the handgrip element; and
for execution of the PDT, the handgrip element, in a state detached from the needle section, serves as a plug element for purposes of insertion into the port on the power supply unit.

13. A light applicator according to claim 1, further comprising a guide element, which is fixedly connected to, or integrated with, the needle section at a proximal end of the needle section and is shaped to correspond to the handgrip element, such that the guide element and the handgrip element are insertable into each other spring-loaded.

14. A light applicator according to claim 1, wherein:
the needle section has a first diameter transverse to the insertion axis;
the handgrip element has a second diameter transverse to the insertion axis; and
the second diameter is many times larger than the first diameter.

15. A light applicator according to claim 13, wherein:
the needle section has a first diameter transverse to the insertion axis;
the handgrip element has a second diameter transverse to the insertion axis; and
the second diameter is many times larger than the first diameter;
the guide element has a third diameter transverse to the insertion axis; and
the third diameter is larger than the first diameter, and smaller than the second diameter.

16. A light applicator according to claim 1, wherein the handgrip element is produced from at least two interconnected parts.

17. A light applicator according to claim 1, further comprising electronics configured for light applicator identification, wherein the electronics for light applicator identification are arranged in the handgrip element.

18. A light applicator according to claim 1, wherein the handgrip element has a tapered portion that is tapered on a distal side of the handgrip.

19. A light applicator according to claim 18, wherein the tapered portion of the handgrip element is configured so as to grip a proximal end of the needle section in a state the handgrip element is coupled to the needle section, for purposes of positioning and/or insertion of the needle section.
